(19) United States
(12) Patent Application Publication
(10) Pub. No.: US 2013/0035497 A1
(43) Pub. Date: Feb. 7, 2013

(54) METHOD OF MANUFACTURING CYCLIC CARBONATE FROM CARBON DIOXIDE

(71) Applicant: Shiey-Shiun HORNG, Kaohsiung (TW)

(72) Inventor: Shiey-Shiun HORNG, Kaohsiung (TW)

(21) Appl. No.: 13/204,111

(22) Filed: Aug. 5, 2011

(51) Int. Cl. C07D 317/08

(52) U.S. Cl. 549/228

(57) ABSTRACT

A method of manufacturing cyclic carbonate with carbon dioxide including the steps of placing solid catalyst in a reaction tube, vaporizing epoxide molecules within a buffer tank to obtain an epoxide vapor, and injecting carbon dioxide into the buffer tank. The carbon dioxide mixes with the epoxide vapor in the buffer tank to obtain an air mixture. The air mixture is then conducted into the reaction tube, where catalysis by the solid catalyst generates cyclic carbonate.

### BACKGROUND OF THE INVENTION

1. Field of the Invention

The present invention relates to a method of manufacturing cyclic carbonate and, in particular, to a method of manufacturing cyclic carbonate from carbon dioxide in a continuous process.

2. Description of the Related Art

Cyclic carbonate is widely used in the manufacturing industry as a raw material in solvents, paint-strippers, and biodegradable products. Cyclic carbonate can also be used in the pharmaceutical industry, in the electronic industry as a solvent in lithium batteries for enhancing the electrical conductivity of lithium, and in the petroleum industry as an antiknock for promoting the stability of petrol.

Formerly, cyclic carbonate was obtained via a phosgenation method, by mixing carbon monoxide and chlorine to obtain phosgene (also known as COCl₂), and by further reacting with phenol or ethanol to obtain cyclic carbonate. For example, cyclic carbonate and hydrochloric acid are generated when bisphenol and phosgene are reacted in an environment containing an alkaline solution and dichloromethane. However, the process of manufacturing cyclic carbonate described above is complicated and risky due to the toxicity of phosgene and dichloromethane. Hence, the phosgenation method for manufacturing cyclic carbonate risks potential environmental pollution and endangers living organisms.

Recently, carbon dioxide has replaced the materials formerly used to manufacture cyclic carbonate, with a cycloaddition of carbon dioxide to epoxide to produce cyclic carbonate. Mainly, the carbon dioxide used in the manufacture of cyclic carbonate is derived from chemical processes such as petrochemical, power generation or metalworking processes. Using CO₂ as a raw material is not only more ecologically friendly, but also economical and convenient.

As described in the study of Kim et al. in 2003, a conventional method for manufacturing cyclic carbonate is a liquid-phased batch reaction. Carbon dioxide from industrial processes is coupled to epoxide under catalysis by an ionic liquid with ZnCl₂ in order to generate cyclic carbonate. The conventional method for manufacturing cyclic carbonate comprises a step of “catalysis,” by preparing a catalyst consisting of 1-butyl-3-methylimidazolium bromide (also called [Bmim]Br) in sticky liquid form; a step of “cyclization,” by mixing the catalyst ([Bmim]Br/ZnCl₂) with propylene oxide in a stainless reactor for coupling the carbon dioxide to alkylene oxide under the catalysis to produce alkylene carbonate; and a step of “isolation,” by isolating alkylene carbonate from the reaction mixture via a distillation method.

Nevertheless, the conventional batch method of manufacturing alkylene carbonate described above is a time-consuming and inefficient process because the reaction mixture is complicate, with the complex catalyst comprising a compound insoluble to alkylene oxide and soluble to cyclic carbonate produced when carbon dioxide and alkylene oxide are reacting. Moreover, the step of “cyclization” is performed in a stainless reactor which requires repeated cleaning after use for another batch.

Finally, the alkylene carbonate requires an additional process of distillation to collect pure alkylene carbonate. The conventional manufacturing process is therefore complicated, time-consuming, and inefficient.

Thus, because of the disadvantages of the conventional method of manufacturing cyclic carbonate, there is a need to provide a new method of manufacturing cyclic carbonate from carbon dioxide.

### SUMMARY OF THE INVENTION

The primary objective of the present invention is to provide a method of manufacturing cyclic carbonate from carbon dioxide in a continuous process, rather than in batches.

The secondary objective of the present invention is to provide a more convenient method of manufacturing cyclic carbonate with carbon dioxide, in which the need for repeated cleaning of the reactor after use is eliminated.

Another objective of the present invention is to provide a method of manufacturing cyclic carbonate from carbon dioxide, which can avoid the disadvantages caused by complicate reacting mixture, so as to be highly efficient.

A method of manufacturing cyclic carbonate with carbon dioxide comprises a step of “placement,” by placing a solid catalyst into a reaction tube; a step of “vaporization,” by vaporizing epoxide molecules within a buffer tank to obtain an epoxide vapor; and a step of “cyclization,” by injecting carbon dioxide into the buffer tank, with the carbon dioxide mixing with the vaporized epoxide in the buffer tank to obtain an air mixture, which is then conducting into the reaction tube, generating cyclic carbonate continuously under fixed-bed catalysis.

Further scope of the applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent in the future.

### BRIEF DESCRIPTION OF THE DRAWINGS

The present invention will become more fully understood from the detailed description given herein below and the accompanying drawings which are given by way of illustration only, and thus are not limitative of the present invention.

FIG. 1 is a diagram illustrating an embodiment of the method of manufacturing cyclic carbonate in the present invention.

FIG. 2 is a FT-IR datum of propylene carbonate in the present invention.

FIG. 3 is a FT-IR datum of a standard sample of propylene carbonate.

FIG. 4 is another diagram illustrating an embodiment of the method of manufacturing cyclic carbonate in the present invention.

FIG. 5 is a FT-IR datum of activated silica gel in the present invention.

FIG. 6 is a diagram further illustrating the formulation of the SilprCl in the present invention.

FIG. 7 is a FT-IR datum of the SilprCl of the present invention.

FIG. 8 is a diagram illustrating the formulation of the Silprlm of the present invention.

FIG. 9 is a diagram illustrating the formulation of the (Bpim)Br/SiO₂.

FIG. 10 is a FT-IR datum of the (Bpim)Br/SiO₂ of the present invention.

FIG. 11 is a TGA datum of silica gel.

FIG. 12 is a TGA datum of the SilprCl of the present invention.

FIG. 13 is a TGA datum of the Silprlm of the present invention.

FIG. 14 is a TGA datum of the (Bpim)Br/SiO₂ of the present invention.

FIG. 15 is a line chart illustrating the conversion of propylene oxide (PO) with respect to reaction temperature as contact time: 20 sec, PO/CO₂: 0.135 and pressure: 20 atm.

FIG. 16 is a line chart illustrating the conversion of propylene oxide with respect to reaction time as temperature: 110° C., PO/CO₂: 0.135 and pressure: 20 atm.

FIG. 17 is a line chart illustrating the conversion of propylene oxide with respect to the ratio of PO/CO₂ as temperature: 110° C., contact time: 22 sec, and pressure: 20 atm.

All figures are drawn for ease of explanation of the basic nature of the present invention only; the extensions of the figures with respect to number, position, relationship, and dimensions of the parts to form the preferred embodiments will be explained or will be within the scope of this patent after the following description of the present invention has been read and understood. Further, the exact dimensions and dimensional proportions to conform to specific weight, strength, and similar requirements will likewise be within the scope of this patent after the following description of the present invention has been read and understood.

### DETAILED DESCRIPTION OF THE INVENTION

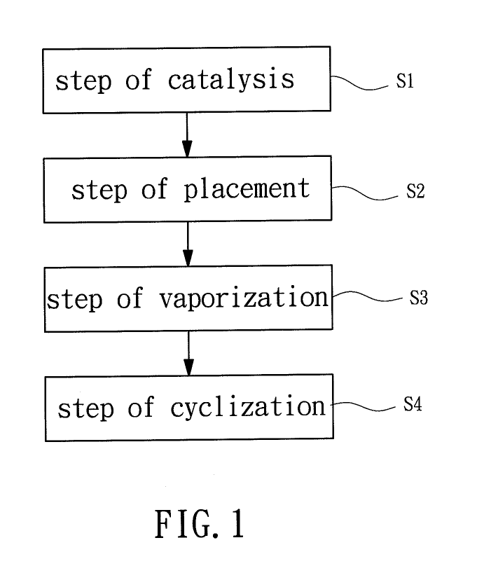

Referring to FIG. 1, in accordance with an embodiment of the present invention, a method of manufacturing cyclic carbonate with carbon dioxide comprises a step of catalysis S1, a step of placement S2, a step of vaporization S3, and a step of cyclization S4.

In the step of catalysis S1, an ionic liquid is prepared and immobilized on a carrier in order to obtain a solid catalyst. In particular, the ionic liquid consisting of anion and cation, is immobilized on the carrier, in order to obtain the ionic solid catalyst.

In the step of placement S2, the solid catalyst is placed within a reaction tube. In particular, the solid catalyst is filled in and closely attached to the reaction tube of a tubular reactor. As an example, 3 grams of a solid catalyst, such as (Bpim)Br/ZnCl₂/SiO₂, is placed into the reaction tube to provide a catalytic fixed-bed.

In the step of vaporization S3, epoxide molecules are vaporized in a buffer tank, preferably in an airtight buffer tank, to obtain an epoxide vapor. In particular, the epoxide molecules, either ethylene oxide or propylene oxide, are heated in the buffer tank for vaporization. As an example, 5 ml of propylene oxide are heated in the buffer tank at >60° C., in order to obtain propylene oxide vapor.

In the step of cyclization S4, carbon dioxide is fed into the buffer tank and mixed with the epoxide vapor in the buffer tank, conducting a cycloaddition reaction via catalysis, thereby obtaining cyclic carbonate. In particular, the carbon dioxide, which can be a gas or a supercritical fluid, is injected into the buffer tank so as to mix with the epoxide vapor. Meanwhile, the reaction tube is heated with a tubular stove and, accordingly, pressure in the reaction will increase as the temperature rises. In this way, the mixture in the buffer tank can flow into the reaction tube due to the difference between the pressure in the buffer tank and the pressure in the reaction tube. Next, cyclization takes place and the cyclic carbonate is produced in the reaction tube. As an example, pressure in the high-pressured gas cylinder is 10-50 atm for injecting the carbon dioxide into the buffer tank, so as to mix the carbon dioxide with vaporized propylene oxide. Moreover, the reaction tube is heated to 90-130° C. to adjust the flow rate of the carbon dioxide to 4-15 ml per minute. Finally the cyclization is performed in the reaction tube to generate propylene carbonate. The detailed chemical reaction of the process in the step of cyclization S4 is summarized in Reaction 1.

C₃H₆O+CO₂→C₄H₆O₃ Reaction 1:

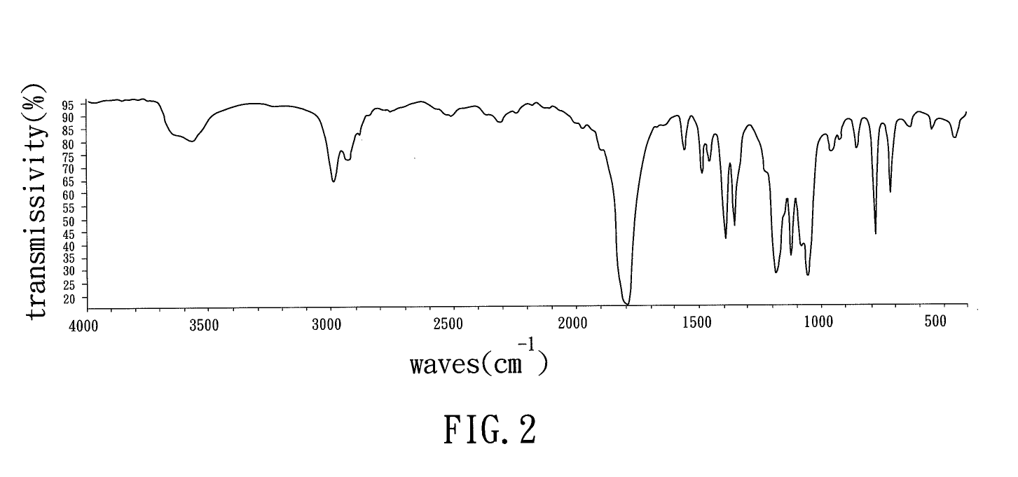

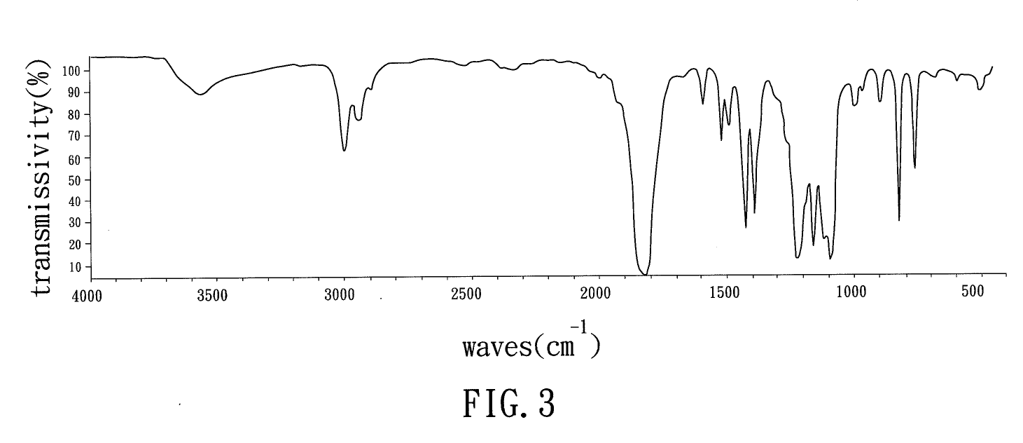

FIG. 2 shows an analyzed datum of Fourier Transform Infrared Spectroscopy (also known as FT-IR) of the propylene carbonate in the present invention, which has a vibrating peak of C═O at 1783 cm⁻¹ and C—O—C at 1310-1000 cm⁻¹. In comparison with a datum of standard propylene carbonate shown in FIG. 3, it is confirmed that the product of the present invention is propylene carbonate.

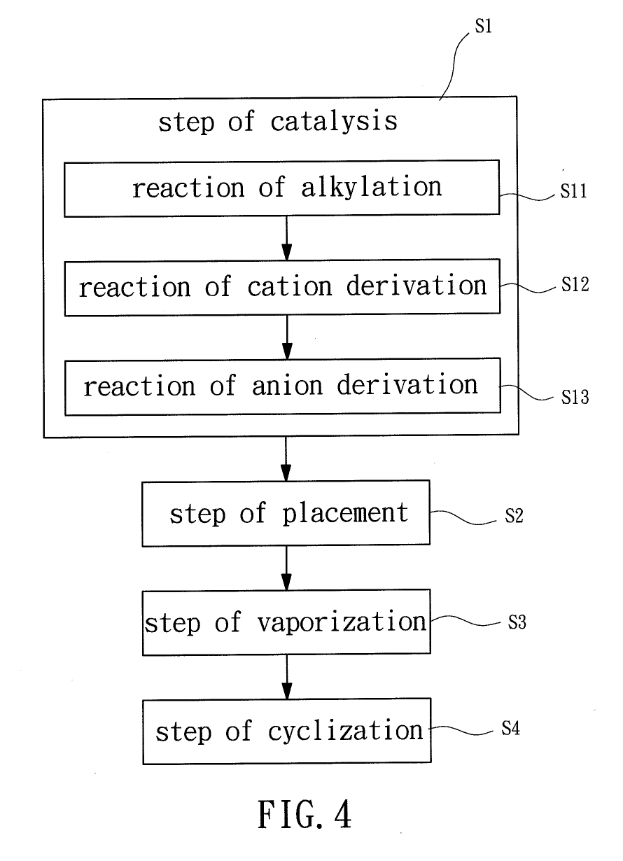

With reference to FIG. 4, to further describe the method of manufacturing cyclic carbonate in the present invention, the step of catalysis S1, further comprises a reaction of alkylation S11; a reaction of cation derivation S12; and a reaction of anion derivation S13. In the reaction of alkylation S11, a carrier, such as silica gel, active carbon, zeolite or other silicic materials, is prepared and alkylated to obtain a haloid carrier. In the reaction of cation derivation S12, the haloid carrier is mixed and interacted with a cationizable compound to obtain a cationizable carrier, which said the cationizable compounds can be alkyl quaternary ammoniums, alkyl quaternary phosphoniums, N-alkyl imidazoliums or N,N-dialkyl pyridiniums. In the reaction of anion derivation S13, the cationizable carrier further interacts with a high polar organic compound to obtain the ionic or ionizable solid carrier of the present invention. The anions can be halides, P⁻² or S⁻² or their oxides or AlCl⁻₄. Finally, to further enhance the activity of the solid carrier, the surfaces of the solid carrier are coated with a layer of Lewis acid, which is an MX compound of a transitional metal. In the preferred embodiment of the present invention, the M can be zinc, manganese, lead, or indium, and the X can be fluorine, chlorine, bromine, or iodine.

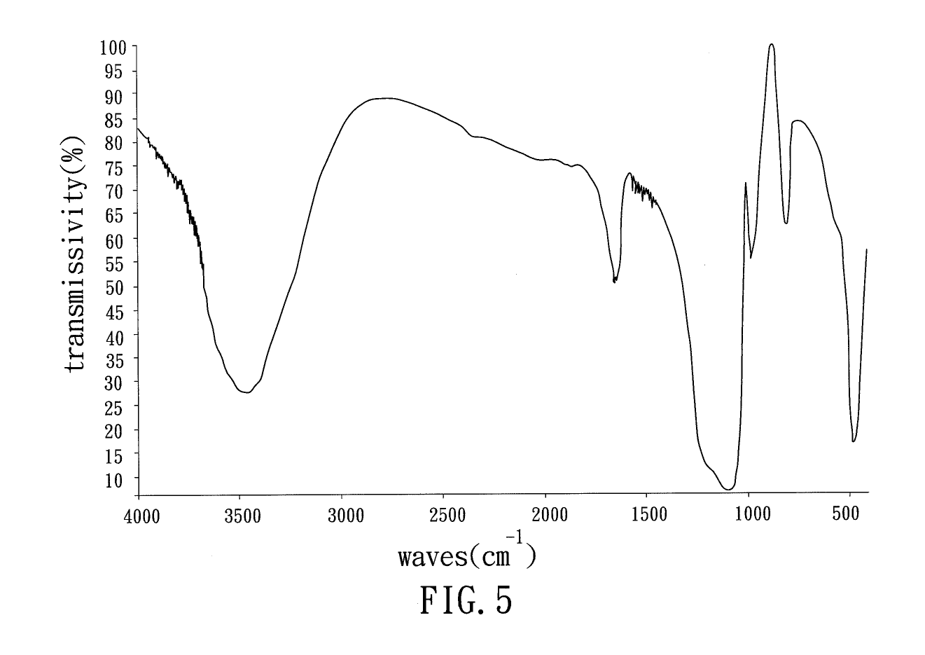

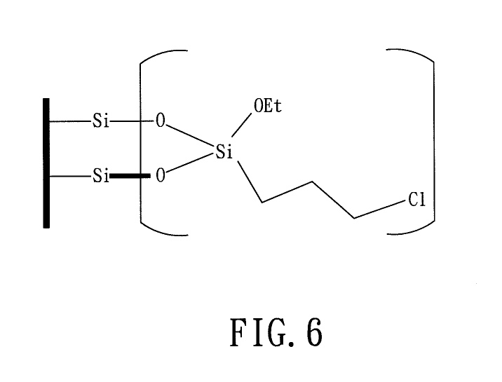

As an example, a silica gel is prepared and activated via an acidification process, by mixing and stirring 15 grams of silica gel and 500 ml of hydrochloric acid (HCl) at room temperature for 1 day; a filtration and then a washing process, by washing with reverse osmosis water; and a drying process, by providing a vacuum condition of 50-60° C. to heat the silica gel for 3-8 hours, sequentially. As shown in FIG. 5, the activated silica gel has been analyzed by FT-IR, which shows clearly vibrating peaks of Si—O at 1000-1200 cm⁻¹, and Si—OH at 1030 cm⁻¹. Then, 5-12 grams of the activated silica gel and 30-125 ml of 3-chloropropyltriethoxysilane are reacted in a flask filled with 250-400 ml of anhydrous toluene. The silica is silanized under catalysis by 2-3 ml of triethylamine. By heating at 120° C. for 48 hours, it is then cooled at room temperature, filtrated, washed with anhydrous toluene and alcohol for removing 3-chloropropyltriethoxysilane, and dried for 3-4 hours, in order to obtain chloropropyl silica (SilprCl, see FIG. 6).

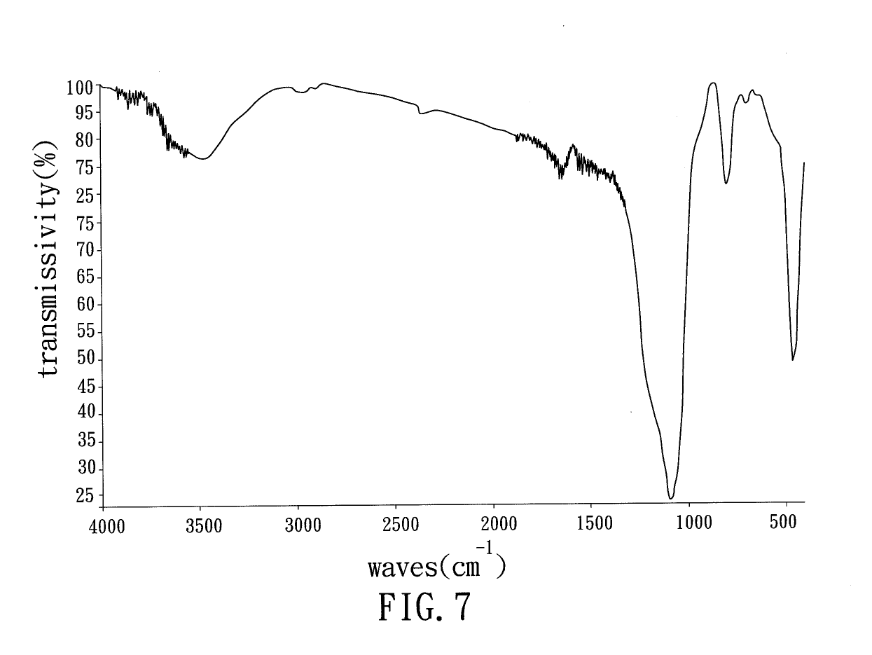

With reference to FIG. 7, in accordance with the analyzed datum of FT-IR, the SilprCl only shows vibrating peaks of Si—C, C—Cl and O—CH₂ after the alkylation, at around 850-650 cm⁻¹, 830-600 cm⁻¹ and 2880-2835 cm⁻¹, respectively.

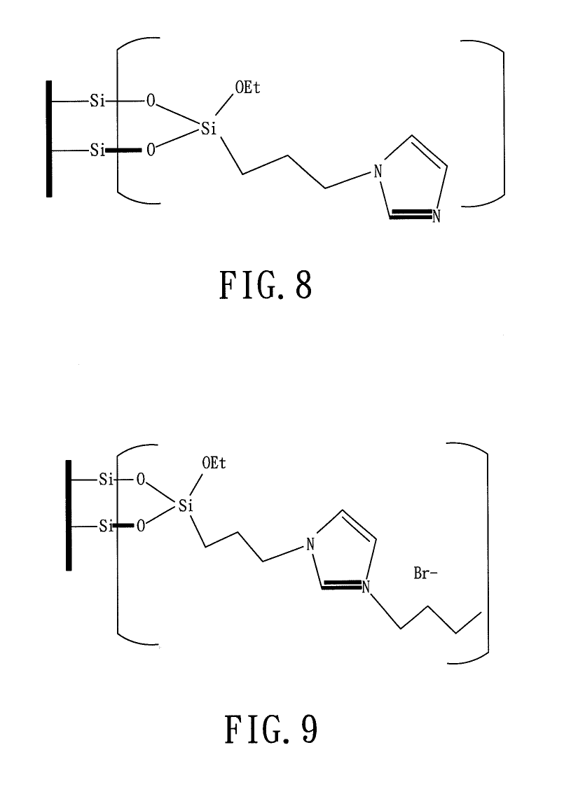

Next, 5-13 grams of SilprCl and 13 grams of imidazolium compounds are reacted in a flask filled with 250-400 ml of anhydrous toluene again. The reaction takes place at 120° C. for 48 hours. The mixture is then cooled at room temperature, filtrated, washed with anhydrous toluene and alcohol, and dried for 3-4 hours, to obtain SilprIm (see FIG. 8).

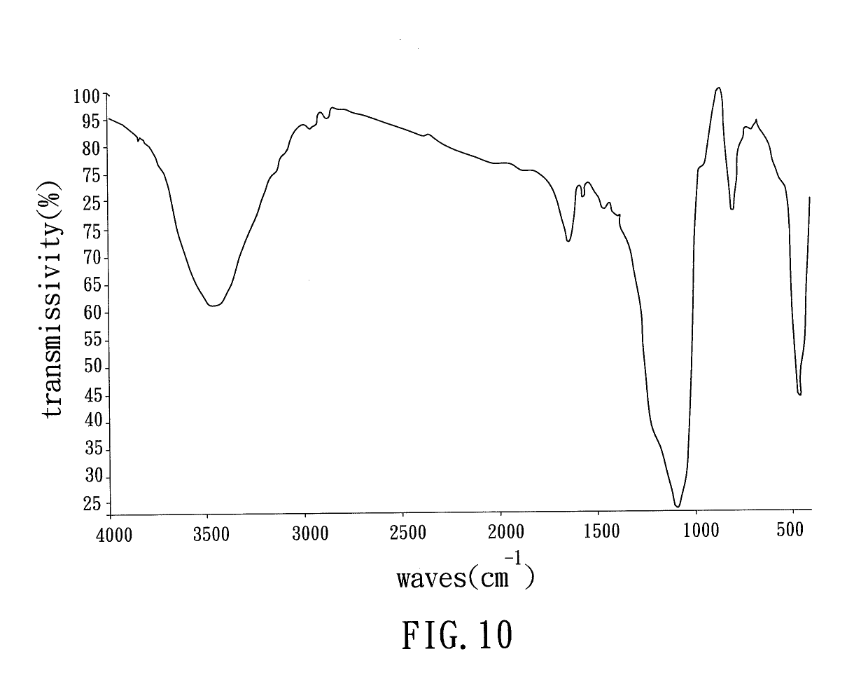

Next, 5-12.3 grams of SilprIm and 60-250 ml of 1-bromobutane are reacted in another flask filled with 250-400 ml of anhydrous toluene. The reaction takes place at 120° C. for 48 hours, and the mixture is cooled at room temperature, filtrated, washed with anhydrous toluene and alcohol, and is dried for 3-4 hours, to obtain (Bpim)Br/SiO₂ (see FIG. 9). With reference to FIG. 10, a FT-IR datum of the (Bpim)Br/SiO₂ shows vibrating peaks of C═C—N and C═N—C, at approximately 1590 cm⁻¹ and 1670 cm⁻¹, respectively.

Furthermore, 5 grams of (Bpim)Br/SiO₂ and 5 grams of Lewis acid (for example ZnCl₂) are mixed and react with each other in a flask filled with 50 ml of tetrahydrofuran (also called THF). The mixture of (Bpim)Br/SiO₂, ZnCl₂ and THF is heated and stirred until the THF is vaporized, in order to obtain a solid compound. The solid compound further undergoes processes of washing with THF, filtration and drying for 3-4 hours, to finally obtain the solid catalyst of the present invention consisting of (Bpim)Br/ZnCl₂/SiO₂.

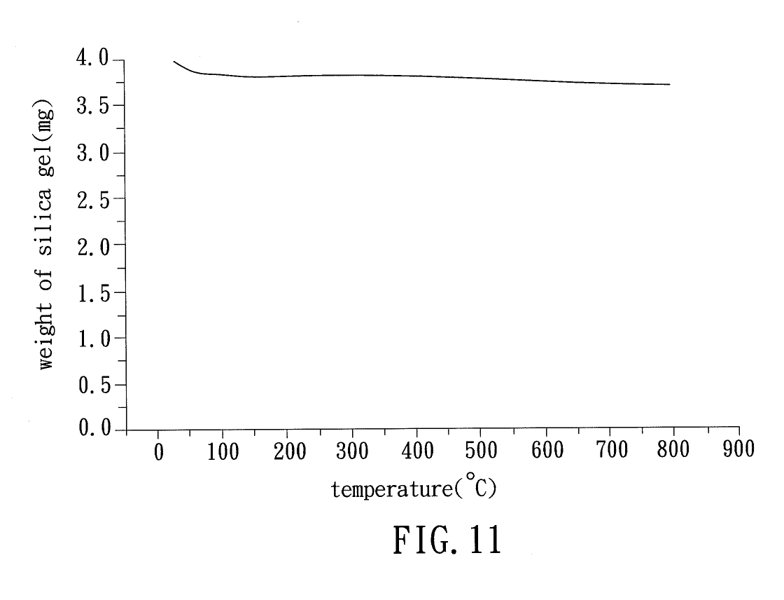

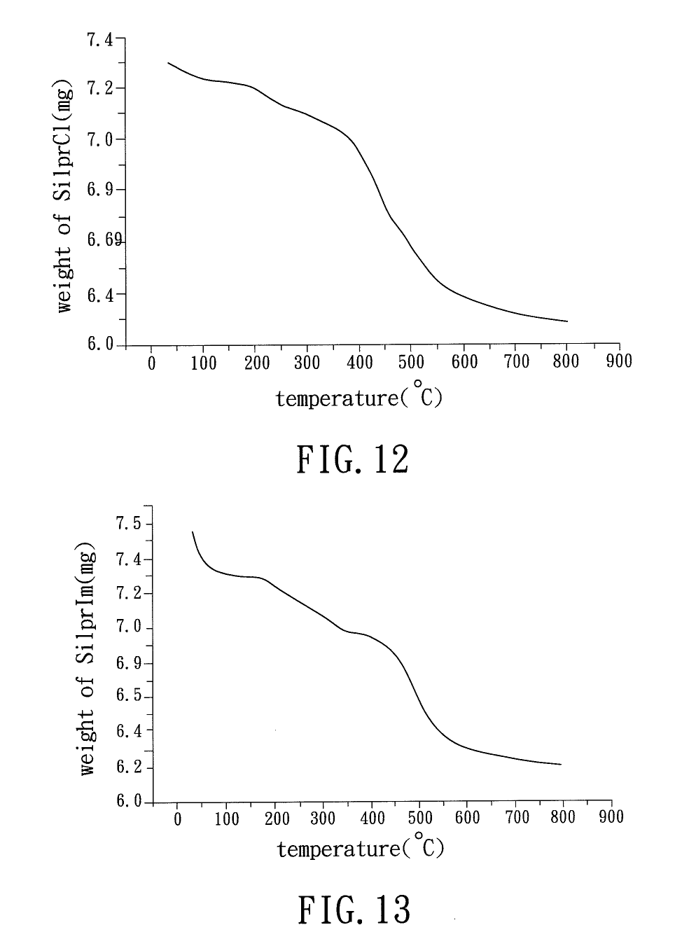

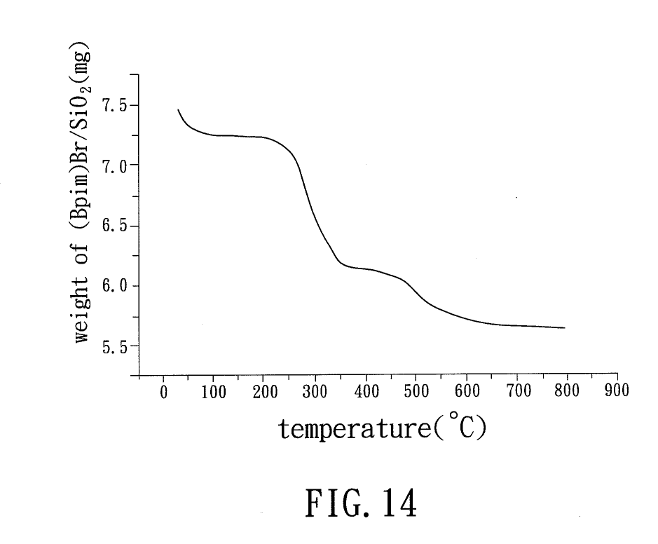

Referring to Table 1, in accordance with the analyzed Thermogravimetry data Analysis (TGA), it is shown that pure silica gel only has a 1.8% loss in weight under a condition of 200-600° C. (with reference to FIG. 11). In contrast, SilprCl, Silprlm and (Bpim)Br/SiO₂ all have obvious losses in weight under a condition of 200-600° C., of approximately 11.4% (with reference to FIG. 12), 12.7% (with reference to FIG. 13) and 20.8% (with reference to FIG. 14), respectively. Therefore, it is proved that the solid catalyst of the present invention comprises multiple layers of alkyl, cation and anion sequentially.

| | Lost Wt. | Lost Wt. | Lost Wt. in |
|---|---|---|---|
| Temperature (° C.) | in SilprCl | in SilprIm | [Bpim]Br/SiO₂ (%) |
| 200~400 | 3.3 | 3.6 | 15.1 |
| 400~600 | 8.1 | 9.1 | 5.7 |
| 200~600 | 11.4 | 12.7 | 20.8 |

In the following section of the embodiment, the efficiency of the method of manufacturing cyclic carbonate is demonstrated by monitoring the conversion of the propylene oxide under different reaction conditions, such as temperature, carbon dioxide pressure, and the ratio of PO and CO₂.

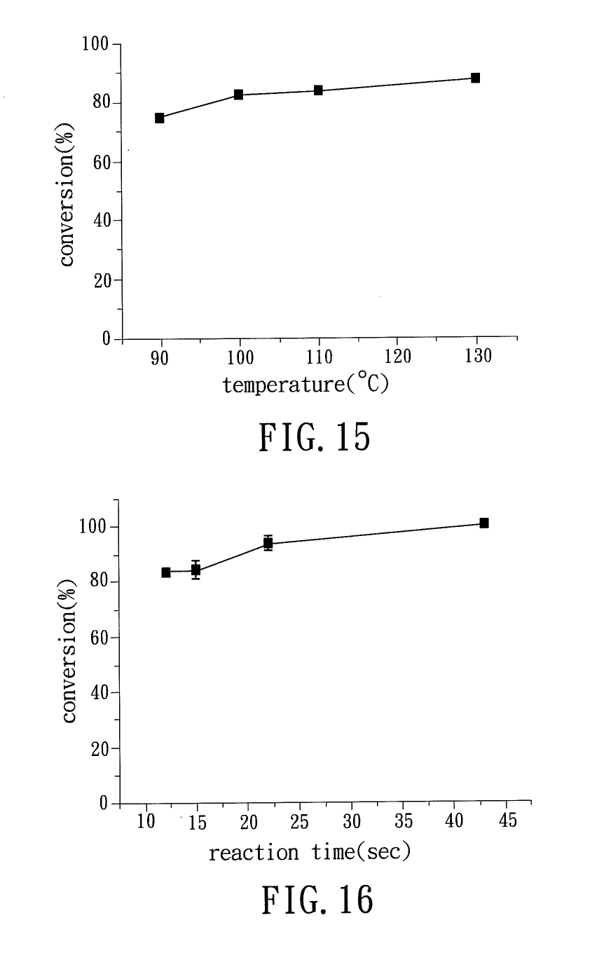

Referring to FIG. 15, manufacture of cyclic carbonate with carbon dioxide is performed with 20 atm of carbon dioxide and 0.135 of PO/CO₂. Also, the contact time of PO and CO₂ with the solid catalyst is set at 12 seconds. In this situation, the conversion of the propylene oxide increases with higher reaction temperature; for example, the conversion is increased from 74.4% to 86.3% when the temperature of the reaction tube goes up from 90° C. to 130° C. Therefore, a higher yield of propylene carbonate can be achieved at higher temperature.

Referring to FIG. 16, the manufacture of cyclic carbonate from carbon dioxide in the present invention is processed at 110° C., with 20 atm of carbon dioxide and 0.135 of PO/CO₂. In this situation, the conversion of the propylene oxide is increased by the prolongation of the contact time of PO and CO₂. For example, the conversion is increased to 100% when the contact time is extended from 12 to 43 seconds. It is suggested that providing a longer contact time for PO and CO₂ to react with the solid catalyst is beneficial to the conversion of propylene oxide to propylene carbonate.

On the other hand, the increase in pressure of carbon dioxide can also advance the conversion of propylene oxide when manufacturing cyclic carbonate with carbon dioxide of the present invention is processed at 110° C. for 22 seconds of contact time. For example, as the pressure increases from 10 to 15, 20, and 25 atm, the conversion of propylene oxide sequentially goes up from 71.3% to 96%. It is suggested that high carbon dioxide pressure can enhance the adsorption of carbon dioxide to the solid catalyst. Therefore, the efficiency of the reaction between CO₂ and PO, as well as the conversion from propylene oxide to propylene carbonate, can be promoted.

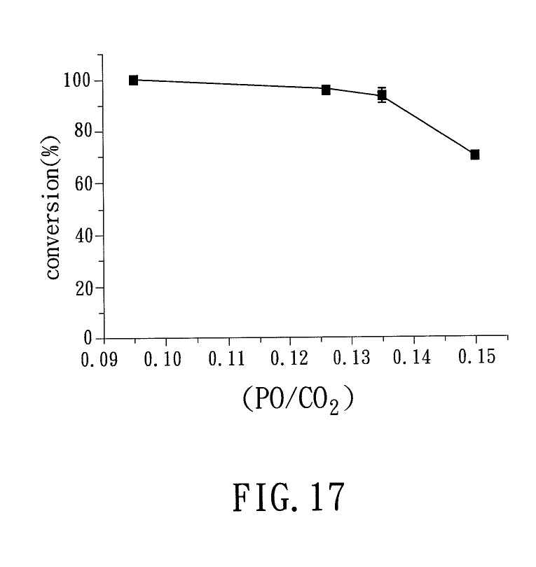

Referring to FIG. 17, the reaction is processed at 110° C., 20 atm for 22 seconds of contact time. In this situation, the conversion of the propylene oxide is decreased by the change of the ratio of PO/CO₂. For example, the conversion varies from 100%, 96.5%, 93.3% to 70.3% when the ratio of PO/CO₂ is increased from 0.095 to 0.126, 0.135 and 0.15. This suggests that the conversion from propylene oxide to propylene carbonate may be interfered with when an improper ratio of PO/CO₂ is provided.

In summary, the method of manufacturing cyclic carbonate in gas phase is beneficial when processed at a proper temperature and pressure, and with a proper ratio of PO/CO₂ and reaction time. In the preferred embodiment of the present invention, the manufacturing method is performed at 130° C. and 25 atm for 43 seconds of reaction time. Accordingly, the ratio of PO/CO₂ can be controlled at 0.135, which makes the conversion from propylene oxide to propylene carbonate a highly efficient process.

Through the invention, a solid catalyst is prepared by immobilizing an ionic liquid, for example [Bmim]Br, on silica gel (SiO₂), so as to obtain a (Bpim)Br/SiO₂. This is followed by coating the (Bpim)Br/SiO₂ with ZnCl₂ to improve the catalyst activity, and finally to obtain (Bpim)Br/ZnCl₂/SiO₂ as the solid catalyst of the present invention. The solid catalyst is placed into a reaction tube for effective catalysis of the reaction between carbon dioxide and propylene oxide. As a result, a high purity of propylene carbonate can be obtained via a simplified continuous manufacturing method, without needing an additional process of purification. Furthermore, the carbon dioxide is mixed with vaporized propylene oxide, which makes the manufacture of cyclic carbonate easily achieved in a continuous process.

Thus, since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

What is claimed is:

1. A method of manufacturing cyclic carbonate with carbon dioxide comprising:
placing ionic or ionizable solid catalyst in a reaction tube;
vaporizing epoxide molecules in a buffer tank to obtain an epoxide vapor; and
performing a cycloaddition reaction, by feeding carbon dioxide into the buffer tank, where the carbon dioxide mixes with the epoxide vapor in the buffer tank to obtain an air mixture, and the mixture flows into the reaction tube, in which cyclic carbonate is generated in a catalytic fixed-bed.

2. The method of manufacturing cyclic carbonate with carbon dioxide as claimed in claim 1, wherein, before the step of placing, an ionic liquid is immobilized on a carrier to obtain the ionic or ionizable solid catalyst.

3. The method of manufacturing cyclic carbonate with carbon dioxide as claimed in claim 2, wherein the carrier is selected from one of silica gel, active carbon, zeolite or other silicic materials.

4. The method of manufacturing cyclic carbonate with carbon dioxide as claimed in claim 2, further comprising a reaction of alkylation, by alkylating the carrier to obtain a haloid carrier; a reaction of cation derivation, by generating a carrier with cationizable groups via an interaction between a cationizable compound and the haloid carrier; and a reaction of anion derivation, by further generating the ionic or ionizable solid catalyst via an interaction between a high polar organic compound and the cationizable carrier.

5. The method of manufacturing cyclic carbonate with carbon dioxide as claimed in claim 1, wherein, before the step of placing, surfaces of the ionic or ionizable solid catalyst are coated with a layer of Lewis acid.

6. The method of manufacturing cyclic carbonate with carbon dioxide as claimed in claim 2, wherein, before the step of placing, surfaces of the ionic or ionizable solid catalyst are coated with a layer of Lewis acid.

7. The method of manufacturing cyclic carbonate with carbon dioxide as claimed in claim 2, wherein the ionic liquid is formulated with a cation selected from one of alkyl quaternary ammoniums, alkyl quaternary phosphoniums, N-alkyl imidazoliums and N,N-dialkyl pyridiniums, and an anion selected from one of halides, P⁻² and S⁻² and their oxides and AlCl⁻₄.

8. The method of manufacturing cyclic carbonate with carbon dioxide as claimed in claim 1, wherein, before the step of cycloaddition reaction, the ratio between the epoxide vapor and the carbon dioxide is set in a value between 0.095 and 2.

9. The method of manufacturing cyclic carbonate with carbon dioxide as claimed in claim 1, wherein, before the step of vaporizing, the buffer tank is heated to 60-100° C.

10. The method of manufacturing cyclic carbonate with carbon dioxide as claimed in claim 1, wherein, before the step of cycloaddition reaction, the pressure of carbon dioxide in the buffer tank is set at 10-50 atm.

11. The method of manufacturing cyclic carbonate with carbon dioxide as claimed in claim 1, wherein the epoxide molecules are selected from one of ethylene oxide or propylene oxide.
